(19) United States
(12) Patent Application Publication
(10) Pub. No.: US 2019/0093166 A1
(43) Pub. Date: Mar. 28, 2019

(54) METHODS FOR CHARACTERIZING THE CELLULAR REPAIR RESPONSE AFTER SOFT TISSUE INJURY

(71) Applicant: RUSH UNIVERSITY MEDICAL CENTER, CHICAGO, IL (US)

(72) Inventors: Anna H.K. Plaas, Chicago, IL (US); Deva Chan, Chicago, IL (US); John D. Sandy, Chicago, IL (US); Vincent Hascall, Chicaggo, IL (US); Katie J. Trella, Chicago, IL (US); Robert Wysocki, Chicago, IL (US); Vincent Wang, Chicago, IL (US); Jun Li, Chicago, IL (US)

(73) Assignee: RUSH UNIVERSITY MEDICAL CENTER, CHICAGO, IL (US)

(21) Appl. No.: 16/081,269

(22) PCT Filed: Mar. 1, 2017
(86) PCT No.: PCT/US2017/020134

§ 371 Date: Aug. 30, 2018

Related U.S. Application Data
(60) Provisional application No. 62/303,760, filed on Mar. 4, 2016.

(51) Int. Cl. C12Q 1/6883; C12Q 1/6806; C12Q 1/686; C12Q 1/6869

(52) U.S. Cl. CPC C12Q 1/6883; C12Q 1/6806; C12Q 1/686; C12Q 2600/16; C12Q 2600/158; C12Q 2600/154; C12Q 2600/112; C12Q 1/6869

(57) ABSTRACT

One aspect of the invention provides a method for classifying the quality of a repair response after injury to a joint of a human or veterinary patient including determining expression levels of at least one of a plurality of genes listed in FIG. 4(A) expressed in a tissue sample taken from the joint.

### RELATED APPLICATIONS

The present patent application claims the benefit of the filing date of U.S. Provisional Patent Application No. 62/303,760, filed Mar. 4, 2016, the contents of which is hereby incorporated by reference.

### STATEMENT AS TO RIGHTS TO INVENTIONS MADE UNDER FEDERALLY SPONSORED RESEARCH AND DEVELOPMENT

This invention was made with Government support of Grant Nos. R01-AR057066 and AR 63144 awarded by the National Institutes of Health. The Federal Government has certain rights in this invention.

### TECHNICAL FIELD

One aspect of the present invention generally relates to methods for determining the quality of recovery from joint injury and to methods of treating joint injury based on such determinations. Another aspect of the invention provides a method for characterizing tendinopathy in a patient. In one embodiment, the method includes detecting a methylation change in a DNA methylome of the patient, where the change is indicative of the tendinopathy.

### BACKGROUND

Aberrant healing, including inflammation and tissue remodeling, is linked to the development of an erosive osteoarthritis (OA) phenotype after traumatic injury, with macrophage activity specifically implicated in the inflammation associated with human knee, hip or hand OA.

### SUMMARY OF THE PREFERRED EMBODIMENTS

In one aspect, the present invention provides a method for classifying the quality of a repair response after injury to a joint of a human or veterinary patient. In one embodiment, the method includes determining mRNA expression levels of at least two of a plurality of genes listed in FIG. 4(A) expressed in a sample from the patient and calculating a reparative index score based on the mRNA expression levels of the at least two of the plurality of genes. Here, the reparative index score is indicative of the quality of the repair process.

The sample may be a tissue sample taken from an intra- or peri-articular region of the joint, for example, a blood sample or synovial fluid aspirate, containing cells.

In another embodiment, the method includes determining mRNA expression levels of at least three, or four, or five, or six, of the plurality of genes listed in FIG. 4(A) expressed in a sample from the patient and calculating a reparative index score based on the mRNA expression levels of the at the least three, or four, or five, or six of the plurality of genes.

Another embodiment provides a method for characterizing tendinopathy in a patient, including detecting a methylation change in a DNA methylome of the patient, where the change is indicative of the tendinopathy. The change may be, for example, a change in a promoter of at least one, two or three of the genes listed in FIG. 18 or FIG. 19 or FIG. 20. The method may include calculating an index score based on the change in the promoter of the genes, where the index score is indicative of the tendinopathy.

Another embodiment provides a method of tendon explant culture methodology allowing for mechanistic studies of tendon cell metabolism independent of contributions from surrounding tissues, while maintaining cells in their native extracellular matrix. The method may include measuring expression of ECM and hypoxia signaling genes during explant culture in High O₂, for example, measuring expression levels of at least two of the genes listed in FIG. 7.

### BRIEF DESCRIPTION OF THE DRAWINGS

FIG. 1 is a photograph showing bone marrow and synovium derived cell cultures

FIG. 2(A-D)—Expression of macrophage markers arginase 1 (Arg1)—FIG. 2A, inducible nitric oxide synthase 2 (Nos2)—FIG. 2B, Mac-1 (Itgam)—FIG. 2C, and F4/80 (Emr1)—FIG. 2D was measured in naïve and injured WT and Has1KO mice and shown as mean (±95% CI) mRNA abundance vs. Gapdh. P<0.05/0.01/0.001 for genotype time point vs. naïve (§/§§/§§§) and WT vs. Has1KO (#/##/###).

FIG. 3(A-B) shows IHC Images of FIG. 3(A) Patellofemoral and FIG. 3(B) Peripatellar Synovium/Subchondral Bone. The location of cells expressing NOS2 and ARG1 proteins was examined by IHC of thin sections of decalcified, paraffin-embedded whole joint sections.

FIG. 4(A-C) are tables showing the effect of macrophage polarization by M-CSF (FIG. 4(B)) and GM-CSF (FIG. 4(C)) on Gene Expression in BM-C and SYN-C Cultures. FIG. 4(A) shown an unstimulated control.

FIG. 5 is a table showing the effect of macrophage stimulation by LPS on Arg1 and Nos2 Gene Expression in BM-C and SYN-C Cultures

FIG. 6 is illustration of an explant culture test method.

FIG. 7 is a table showing fold change in expression of ECM genes.

FIG. 8 is a graphical illustration of the percentage of hypoxia signaling genes up or down-regulated.

FIG. 9 is a table showing the effect of experimental conditions on expression of hypoxia signaling genes

FIG. 10 is a graph showing NADH/NADPH reducing equivalents.

FIG. 11 is a graph showing medium glucose concentration during explant culture.

FIG. 12 is a schematic illustration showing changes occurring in tendinopathy.

FIG. 13 is a schematic illustration showing epigenetic modifications.

FIG. 14 shows a murine model of Achilles tendinopathy.

FIG. 15 illustrates expression of epigenetic enzymes.

FIG. 16 illustrates a methylome analysis model

FIG. 17 illustrates methylation status of promotor regions.

FIG. 18 shows genes with promotor methylation changes.

FIG. 19 shows methylation effects on transcription.

FIG. 20 shows methylation effect on genes relevant to tendinopathy.

FIG. 21 includes data illustrating the stress response of murine Achilles tendons.

### DETAILED DESCRIPTION OF THE PREFERRED EMBODIMENTS

### Definitions

All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification otherwise claimed. No language in the specification should be construed as indicating any non-claimed element as essential to the practice of the invention.

As used herein, the term “patient” refers to a human or veterinary patient. In one embodiment, the veterinary patient is a mammalian patient.

Methods for Characterizing the Cellular Repair Response after Soft Tissue Injury

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

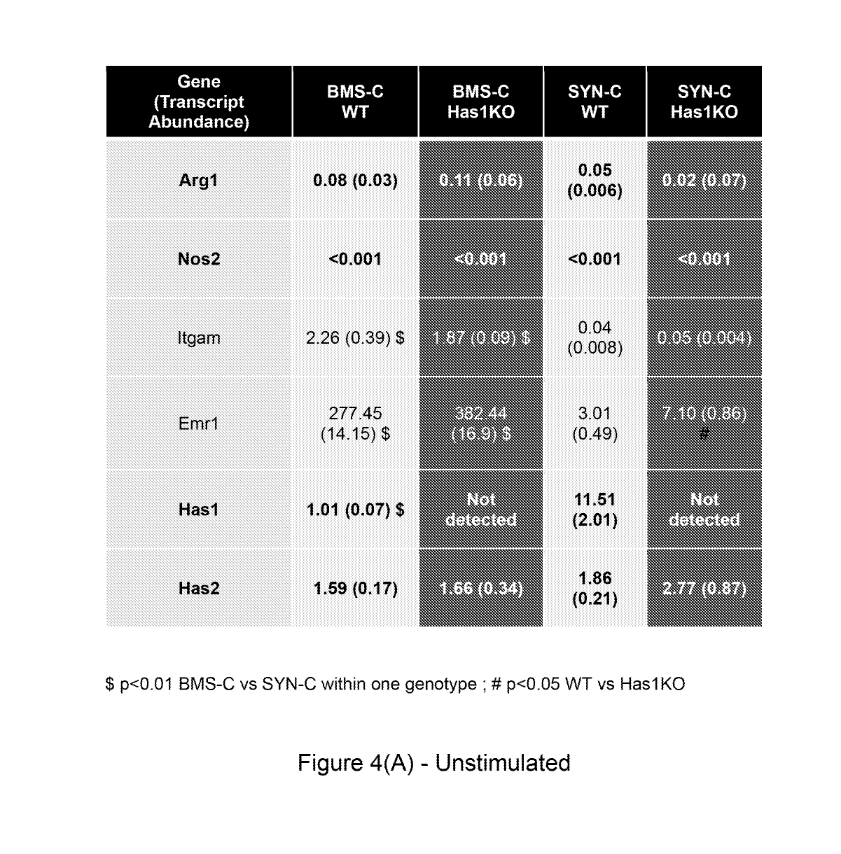

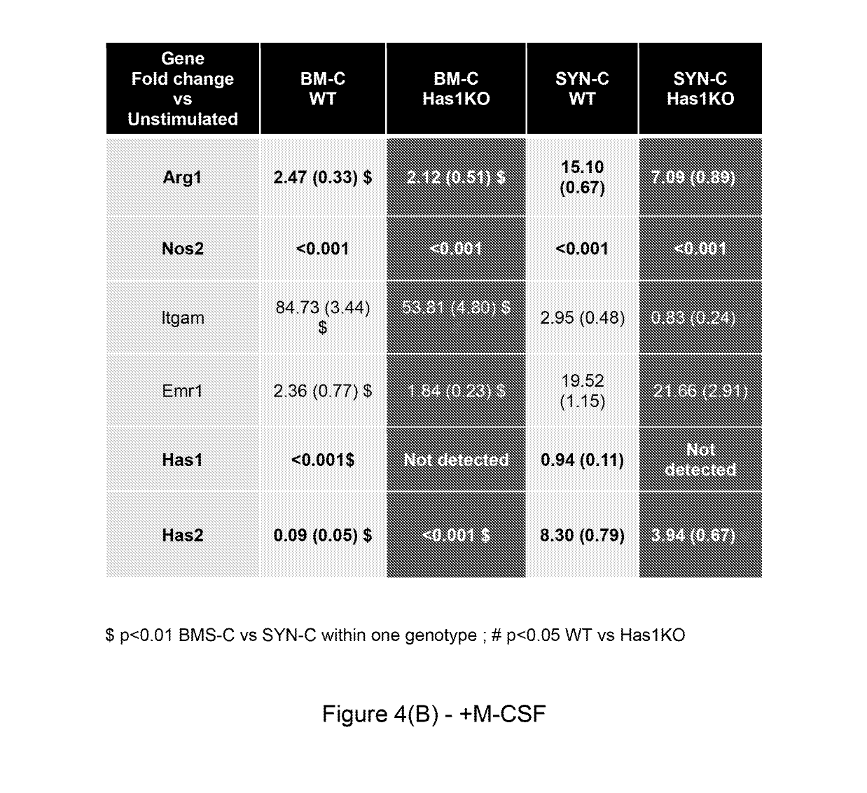

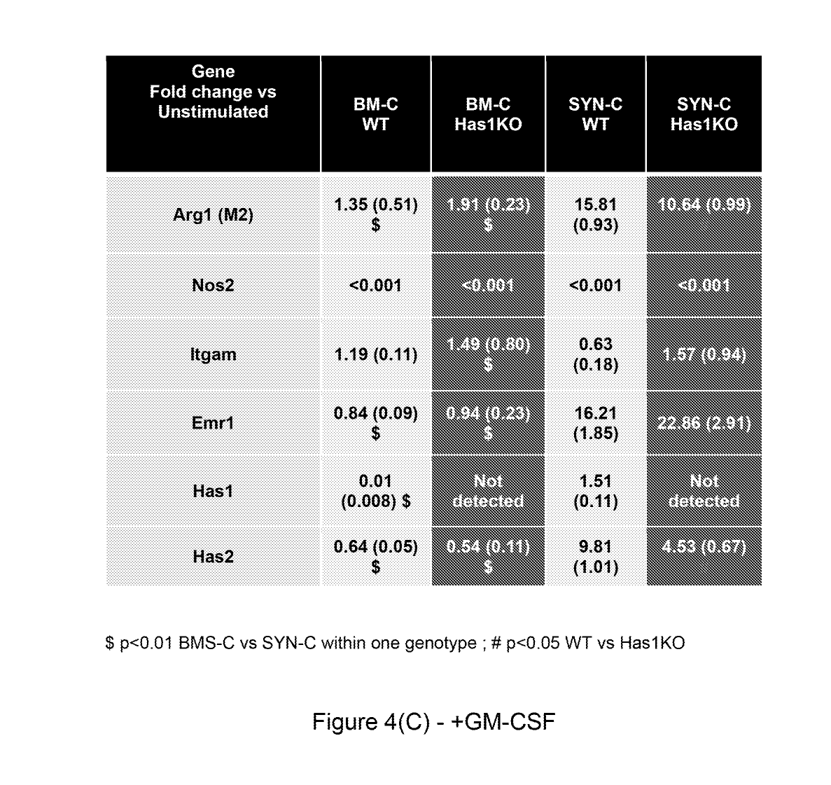

In one aspect, the present invention provides a method for characterizing the quality of the repair response after injury to a joint of a human or veterinary patient. The method includes determining expression levels of at least one, or two, or three, or four, or five, or six of the plurality of genes expressed in a tissue sample taken from an intra- or peri-articular region of the joint. In one embodiment, the plurality of genes includes at least the genes listed in FIG. 4(A), i.e. Arg1, Nos2, Itgam, Emr1, Has1 and Has2. A reparative index score indicative of the quality of the repair response is calculated based on the expression levels of these genes.

In one embodiment, the expression levels are determined using a Reverse Transcriptase-Real Time PCR array assay. The tissue sample can include cartilage, synovium, meniscal tissue, joint capsule lining, ligaments, bone marrow or synovial fluid derived cells or peripheral blood cells or combinations of at least two of these materials.

In certain embodiments, calculating the reparative index score includes comparing the mRNA expression levels from the patient tissue with first standard expression levels of the genes and second standard expression levels of the genes, where the first standard expression levels are indicative of a reparative profile and where the second standard expression levels are indicative of a non-reparative profile. The reparative index score is based on relative values of the mRNA expression levels from the patient tissue, the first standard expression levels and the second standard expression levels.

The first standard expression levels can be post-injury expression levels from a wild-type mouse and the second standard expression levels can be post-injury expression levels from a mouse lacking hyaluronan synthase-1. The method can also include applying a correction factor to adjust the relative abundance of the murine levels with respect to the genes expressed in the patient tissue sample.

In certain embodiments, the patient is a human patient and the tissue sample is obtained at between 4 weeks and 20 weeks, 4 weeks and 16 weeks, 4 weeks and 12 weeks or 6 weeks and 10 weeks after the injury. In other embodiments multiple tissue samples are obtained, each at a different time after the injury. Such a procedure allows the quality of the repair response to be monitored as time progresses. For example, the quality of natural healing of the joint or the response of the injury to a particular treatment can be determined by observing the changes in gene expression over time. For example, the tissue sample can be obtained during the first arthroscopic (or open joint) evaluation of the injury. In certain cases, depending on the surgeons preferences, a second sample could be obtained at a later time (such as at surgery or at a second arthroscopic evaluation)

Another aspect of the present invention provides methods for treating an injury to a joint of a human or veterinary patient including administrating a therapy depending on the reparative index score as discussed above. In certain embodiments, the therapy is surgical reconstruction, physical therapy, viscosupplementation HA therapy, diet recommendations or life-style change recommendations. In other embodiments, the treatment includes administration of anti-inflammatory creams, gels or sprays, heat and freeze treatments, non-steroidal anti-inflammatory drugs (NSAIDs), acupuncture, complementary and alternative medicines, steroid injections or steroid tablets.

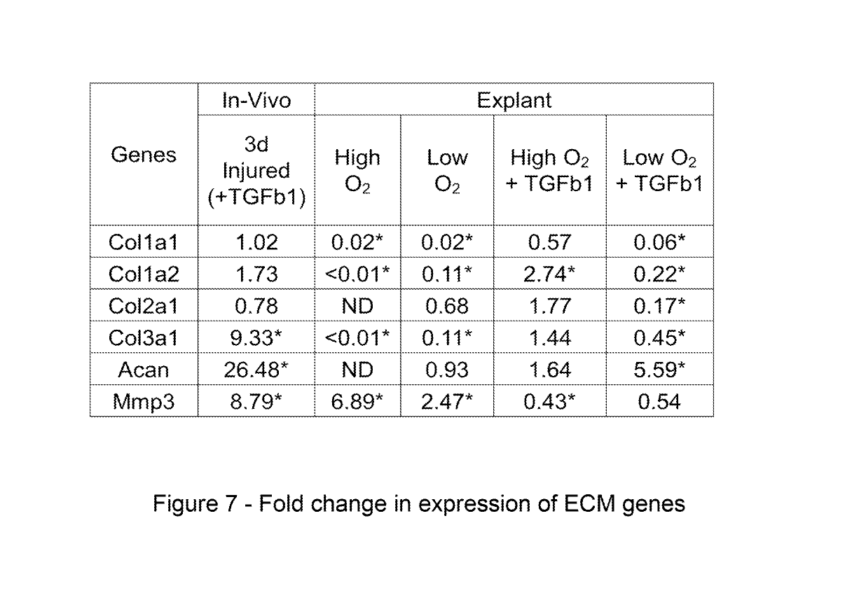

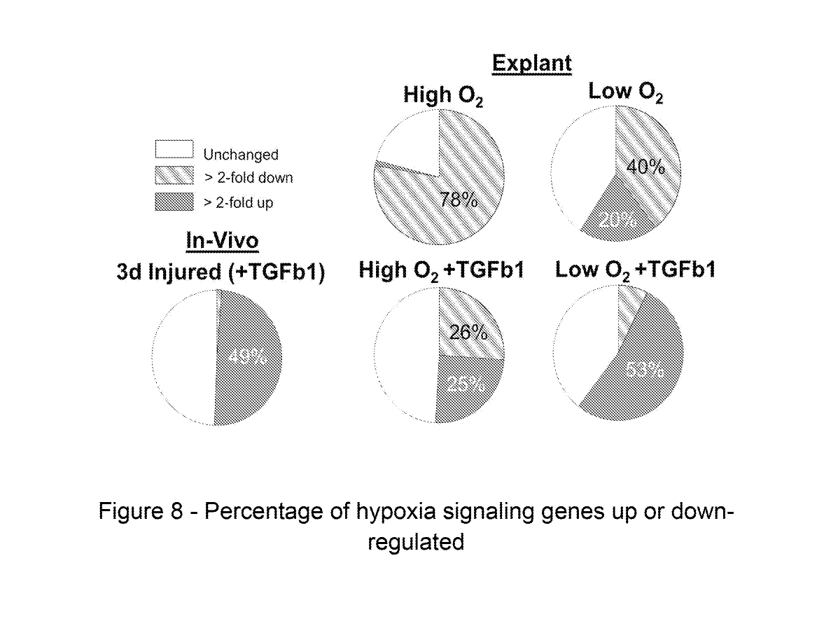

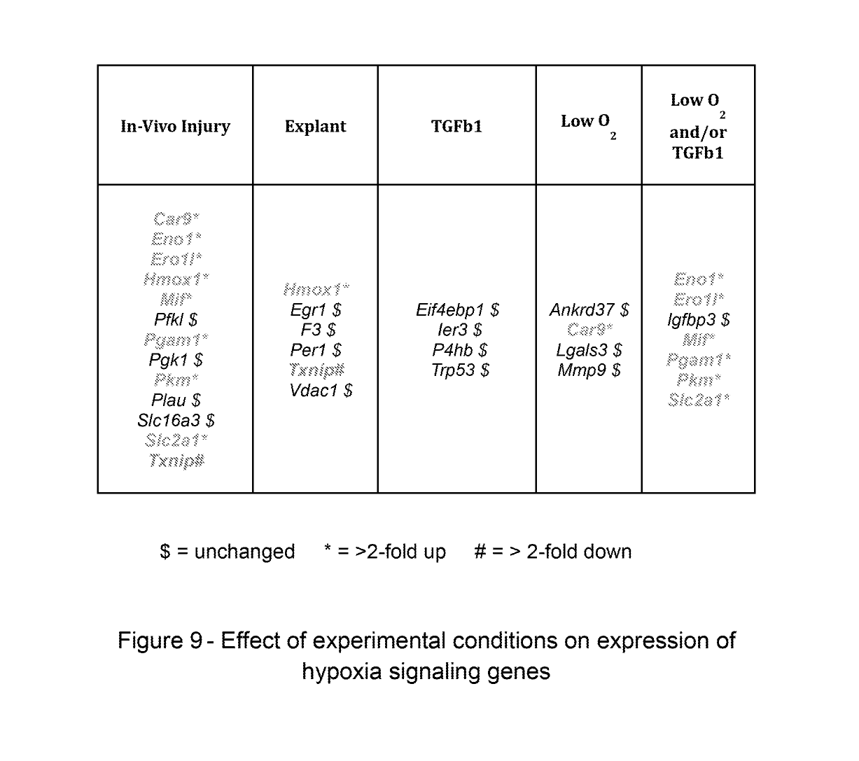

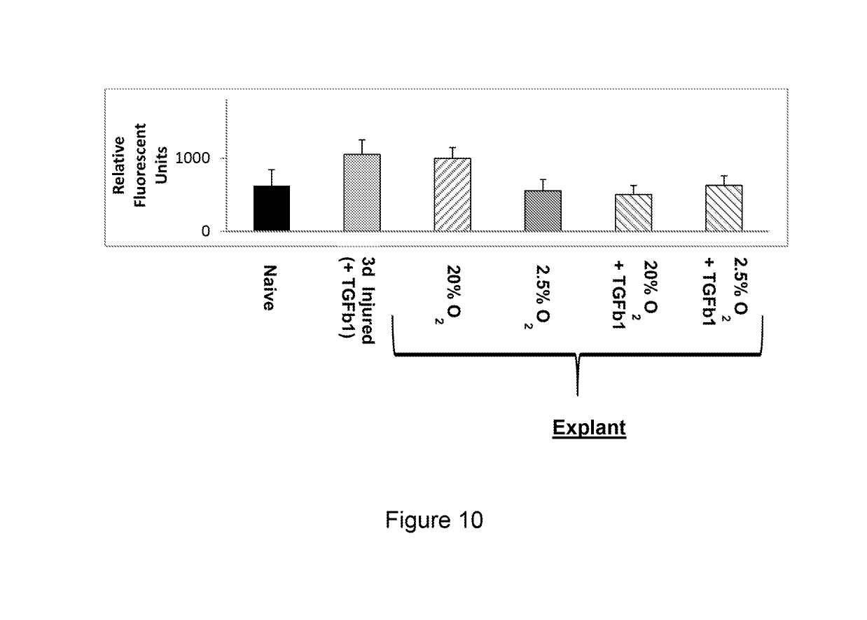

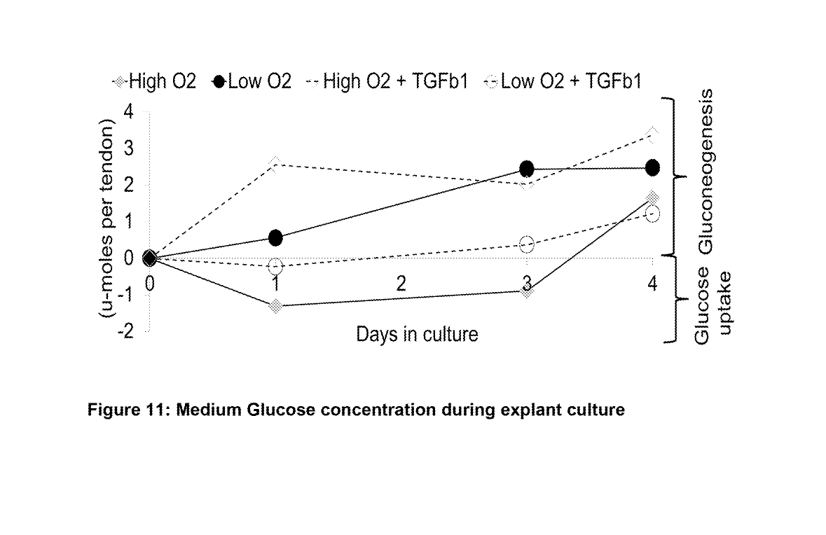

Another aspect of the invention provides an Achilles tendon explant culture method that can be used to test therapeutic modulation for prevention of long-term pathological remodeling of the tissue leading to chronic tendon injury. In one embodiment, the method is utilized to show a reduction in expression of ECM and hypoxia signaling genes during explant culture in High O₂ is prevented by Low O₂ and/or TGFb1 (FIG. 7). This illustrates the altered metabolism of tendon cells after acute tendon injury in-vivo and the activation of metabolism genes in explant with Low O₂ and/or TGFb1 mimics early in-vivo injury (FIGS. 8 and 9. Culture in high O₂ is shown to modify metabolic outcomes (glucose uptake and NADH/NADPH reducing activity) to that of injured tendons (FIGS. 10 and 11.)

Another aspect of the invention provides a method a utilizing methylation changes to characterize tendinopathy in patients. Tendinopathy is a complex disease as a result of age, genetic predisposition, comorbidities, adverse drug effects, and overuse or traumatic injuries. As you can see from FIG. 12, showing the histological sections of normal vs tendinopathic specimens, tendinopathy is characterized by collagen degeneration and chondroid metaplasia. This disruption of the normal extracellular matrix ultimately results in mechanical weakening. Due to the complexity of the disease, successful drug-based and physical therapies are lacking.

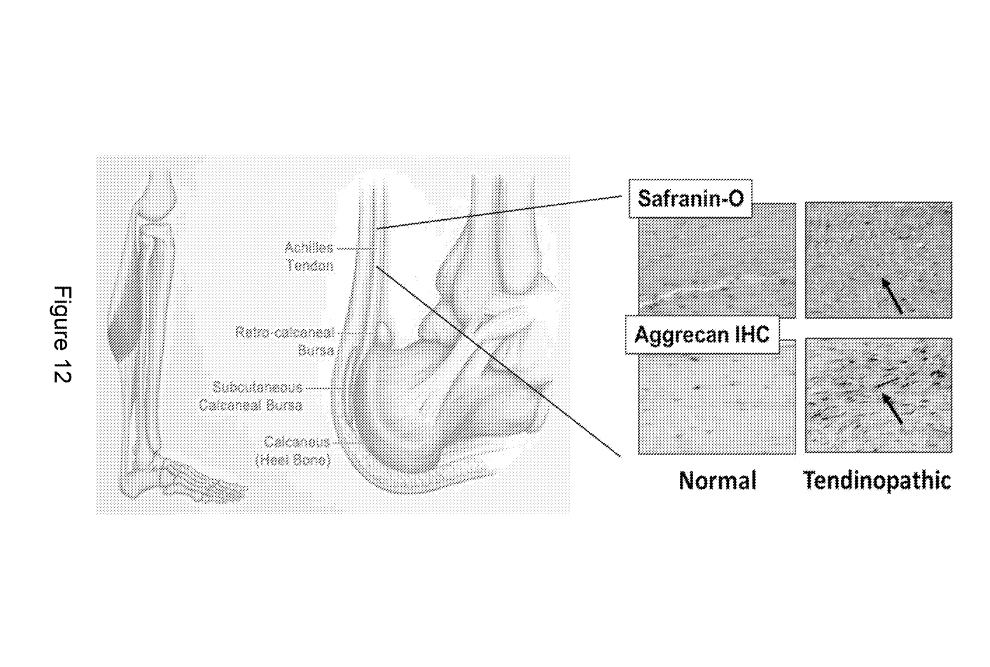

Epigenetics involves chemical modifications to the DNA and histone proteins that the DNA winds around as well as expression of regulatory RNAs. FIG. 13 illustrates that modifications include methylation to the DNA at cytosine sites as well as methylation, acetylation, phosphorylation, and ubiquitation to the histone proteins. The reverse reactions are also possible. These modifications affect the ability of the DNA to wind around these histone proteins. In an un-wound state the DNA is accessible to transcriptional machinery in promoter and enhancer regions of the DNA to promote gene transcription. Recent approaches have used epigenetics to study the mechanisms and therapeutics in complex diseases such as cancer, diabetes, and chronic kidney disease. It has been shown that cells maintain specific epigenetic identities even following cell division, and these epigenetic blueprints may contribute to the chronicity of a disease.

We sought to examine whether chronic tendinopathy is regulated by the epigenome. To do this we used a murine model of tendinopathy to examine temporal changes in the (1) expression of epigenetic enzymes (2) DNA methylome (3) downstream effects of altered DNA promoter methylation on changes in gene expression and (4) the identification of modified genes relevant to tendinopathy.

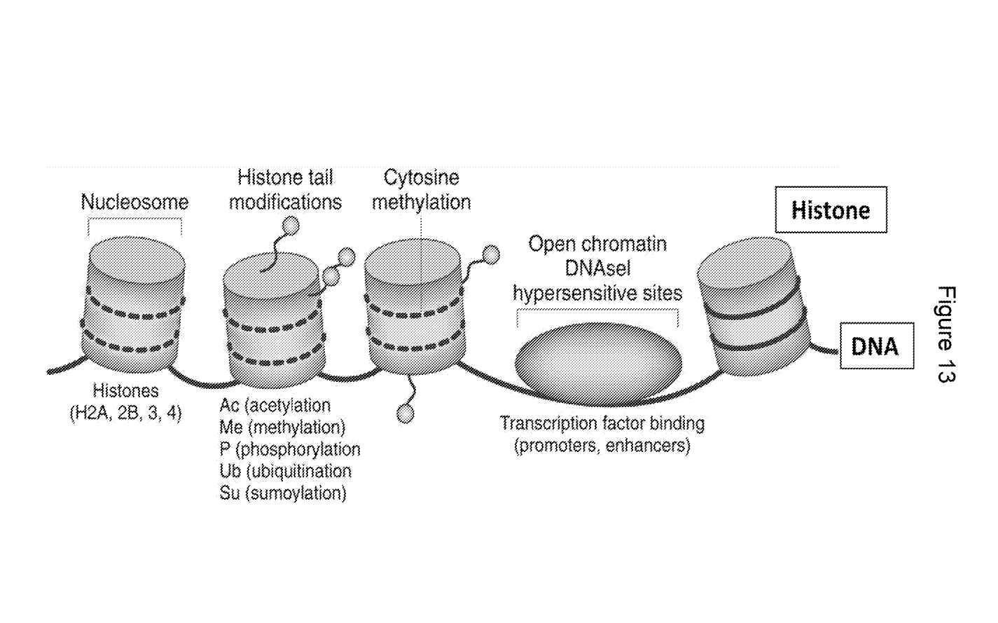

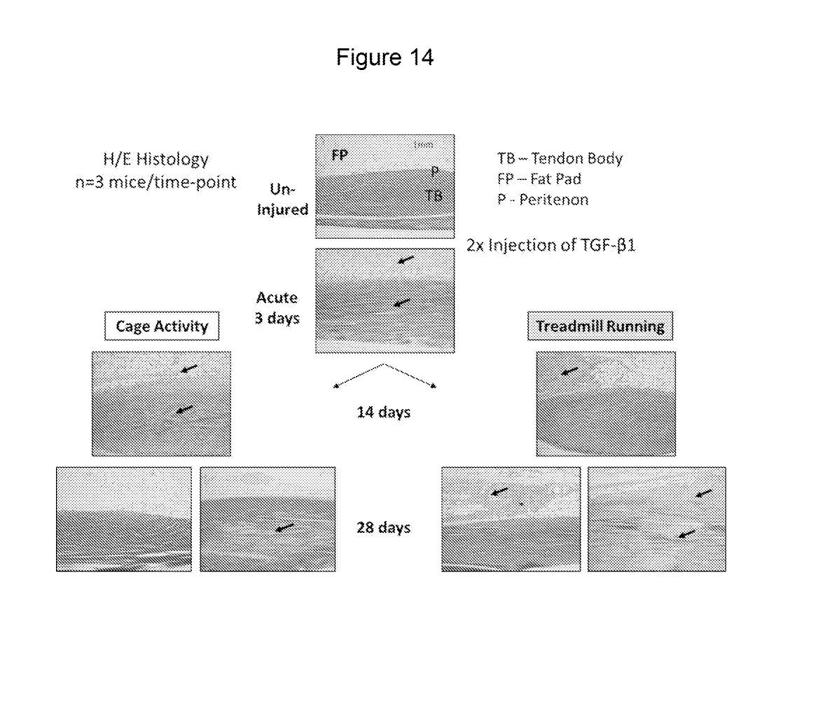

The model, which is illustrated in FIG. 14, induces tendinopathy by injecting TGF beta 1 directly into the body of the Achilles tendon. As early as 3 days post-injury you can see collagen disorganization as well as hyperplasia in the surrounding synovial tissue. Mice can then be maintained with cage activity alone where hyperplasia is seen in the tendon body, peritenon, and surrounding synovial fat pad. Mice can also be run on the treadmill and a more severe overuse phenotype is achieved. A robust and persistent hyperplasia response is seen in the surrounding synovial tissue and peritenon. These injury characteristics are accompanied by prolonged expression of chondrogenic and injury markers as well as result in a chronic loss of material properties.

The method utilized a chromatin modification enzyme QPCR array. For this analysis murine Achilles tendons were removed from surrounding synovial and fatty tissue as seen in the histological section in FIG. 15. To look at the expression of epigenetic enzymes, the 82 genes on the array plate were separated based on modification type; whether they affected the DNA, histones, or either the DNA or histones. The percentage of genes significantly altered from un-injured levels is shown in the table in FIG. 15 for all time-points. Overall, there are changes in the expression of epigenetic enzymes in this model specifically at the 3 day acute time point in response to TGF beta 1 and with treadmill running at 14 days. The remainder of this study focused on DNA methylation.

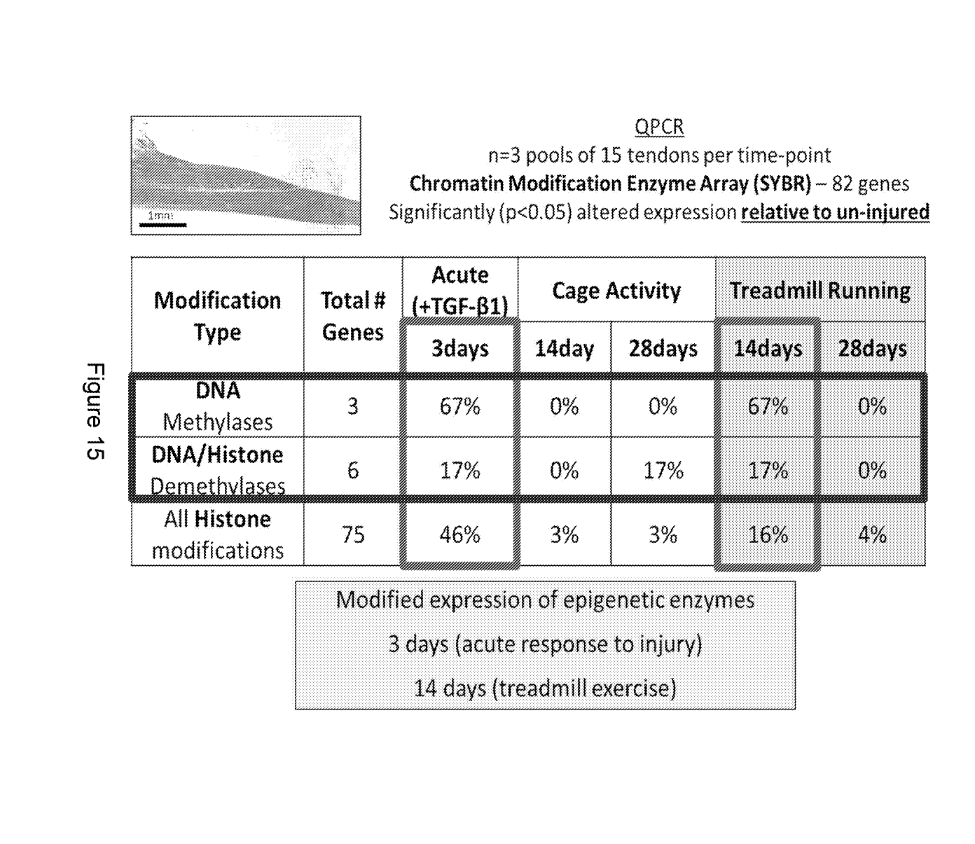

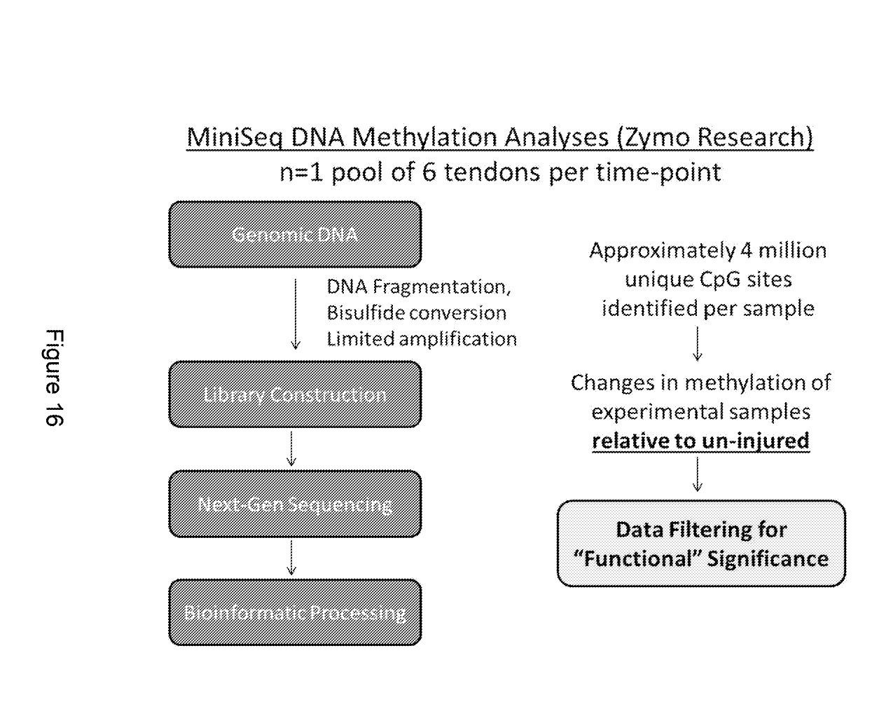

DNA methylome analysis was conducted. Here, genomic DNA was isolated from pools of Achilles tendons for each experimental group. The DNA then undergoes fragmentation, bisulfide conversion, and limited amplification so that a DNA library is constructed for each specimen. See FIG. 16. Next each specimen undergoes next-gen sequencing (NGS) and bioinformatic processing to determination the methylation levels of sites throughout the DNA library. In total approximately 4 million unique sites were identified per sample. We then identified changes in methylation of our experimental samples relative to our un-injured controls and used data filtering to find functional significance.

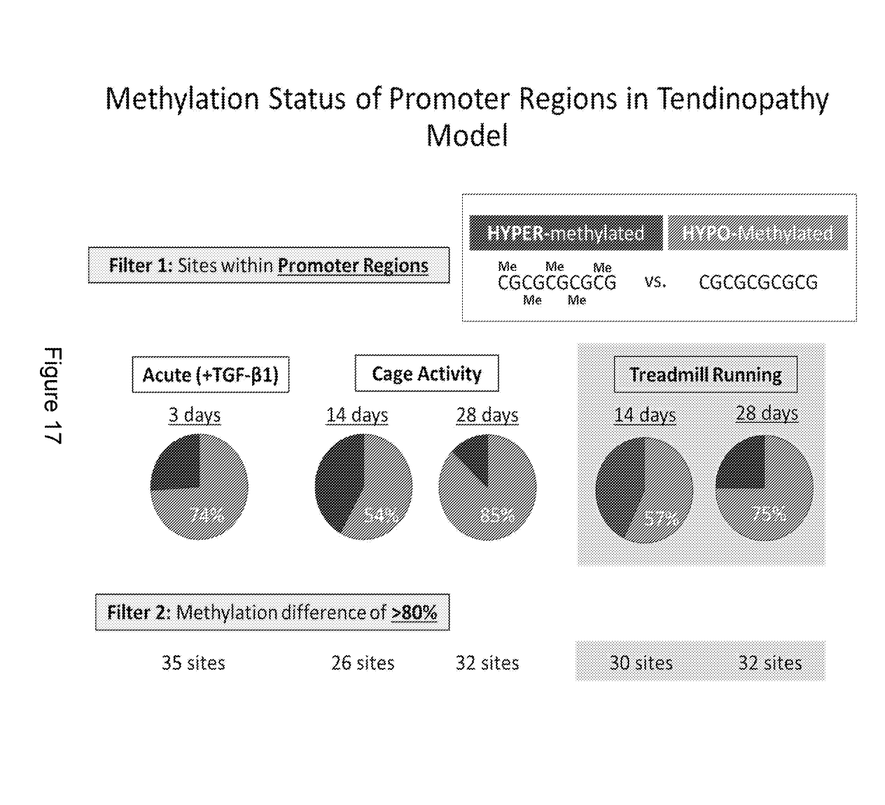

First, we filtered the data so we were only looking at methylation sites within the promoter regions of DNA. Each site can either be hyper-methylated or hypo-methylated relative to un-injured. Looking at the methylation status of all DNA promoter regions it is evident that there is generally hypo-methylation or a decrease in the methylation of DNA with injury, suggesting hypomethylation may be involved in disease chronicity. The data are then filtered to focus on methylation differences of greater than 80% relative to un-injured to ensure that substantial methylation changes are evident. When we applied this filter to all 5 experimental groups we see approximately 30 significantly differentially methylated sites per specimen identified. See FIG. 17.

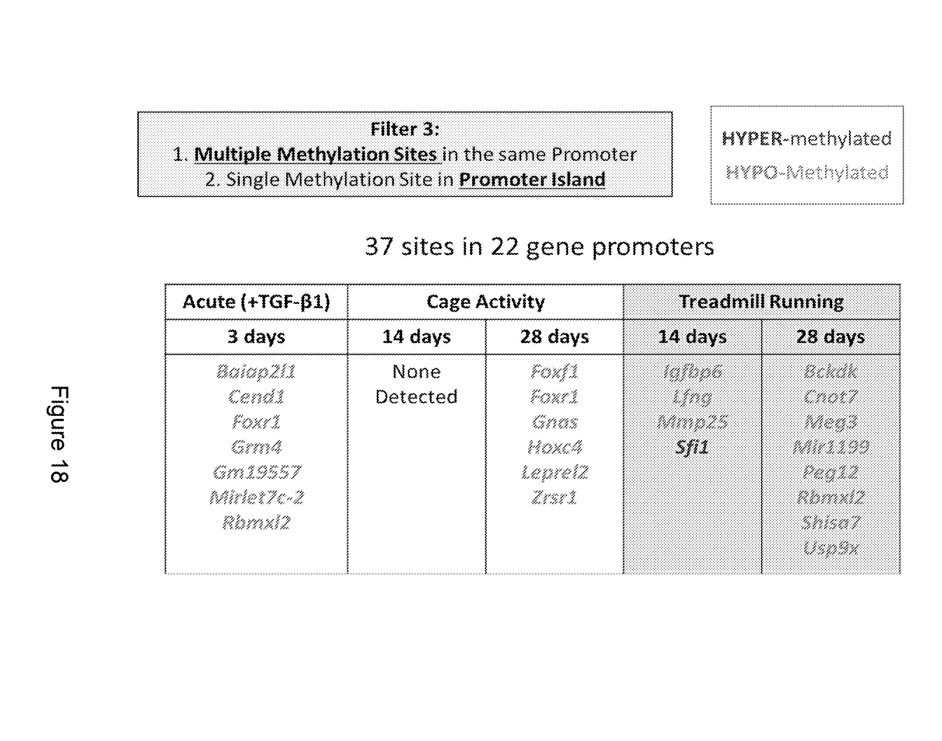

For the 3rd filter, we focused on sites which exhibited multiple significant methylation events within the same gene promoter OR which exhibited significant methylation within promoter island of genes. From this we identified 37 sites in 22 gene promoters. The gene promoters are listed in FIG. 18 and the colors denote if they were hypo-methylated or hyper-methylated relative to un-injured. All genes with the exception of Sfi1 are hypo-methylated. Sfi1 is hyper-methylated. We observe a consistent response in the number of gene promoters affected at all groups except at 14 days with cage activity, and that most of these gene promoters are hypo-methylated. Also note that 2 genes actually show up independently in 2 different groups. These include Foxr1 and Rbmxl2.

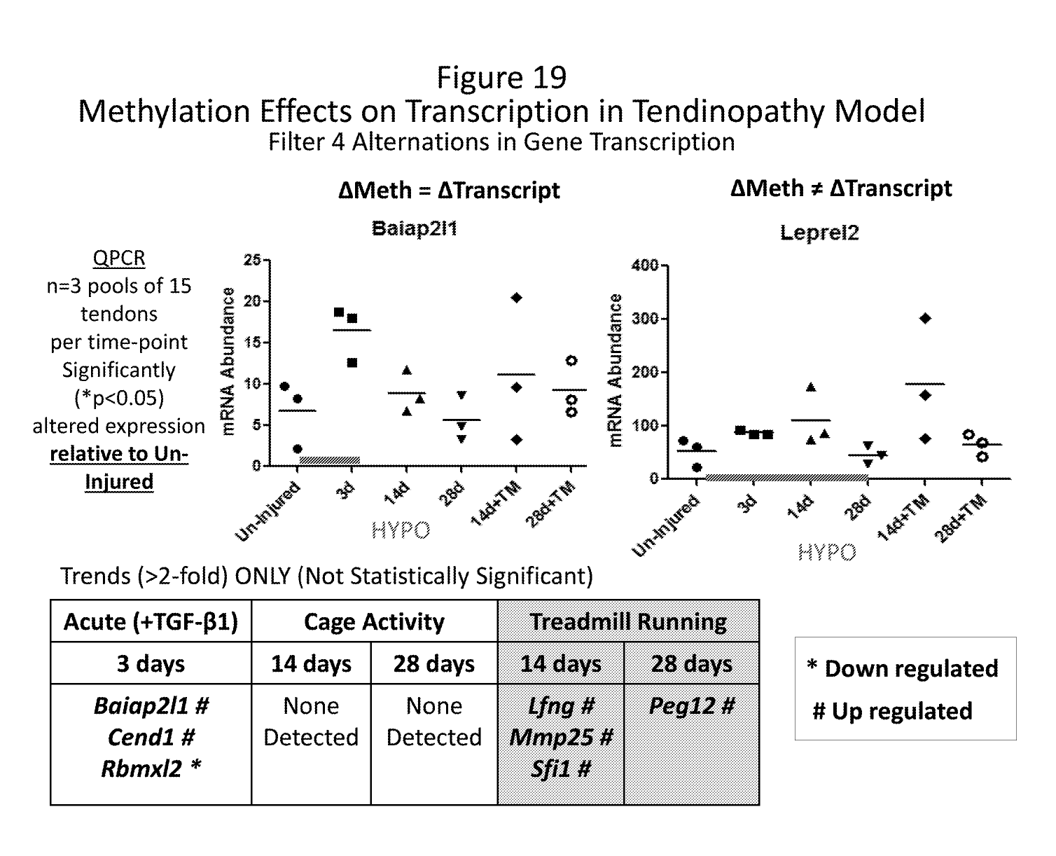

From there we determined if the DNA methylation changes in these gene promoters effected transcription of that gene. As can be seen from the example mRNA abundance scatter plots in FIG. 19, we either saw that a change in methylation correlated with a change in transcription as with Baiap2l1 which was hypomethylated at 3 days and an increase in transcription is seen at 3 days. Or we saw that a change in methylation did not change transcription as with Leprel2, which was hypomethylated at 28 days and no change in expression was observed. However, it is also important to note that expression for some genes changed in the model irrespective of methylation changes. When we applied this to all the genes identified we saw that the expression of 7 genes was either up or down-regulated by methylation to the DNA. However, these are just trends because no gene exhibited statistically significant changes in transcription throughout the injury time-course due to variations between the pools as you can see in the scatter plots.

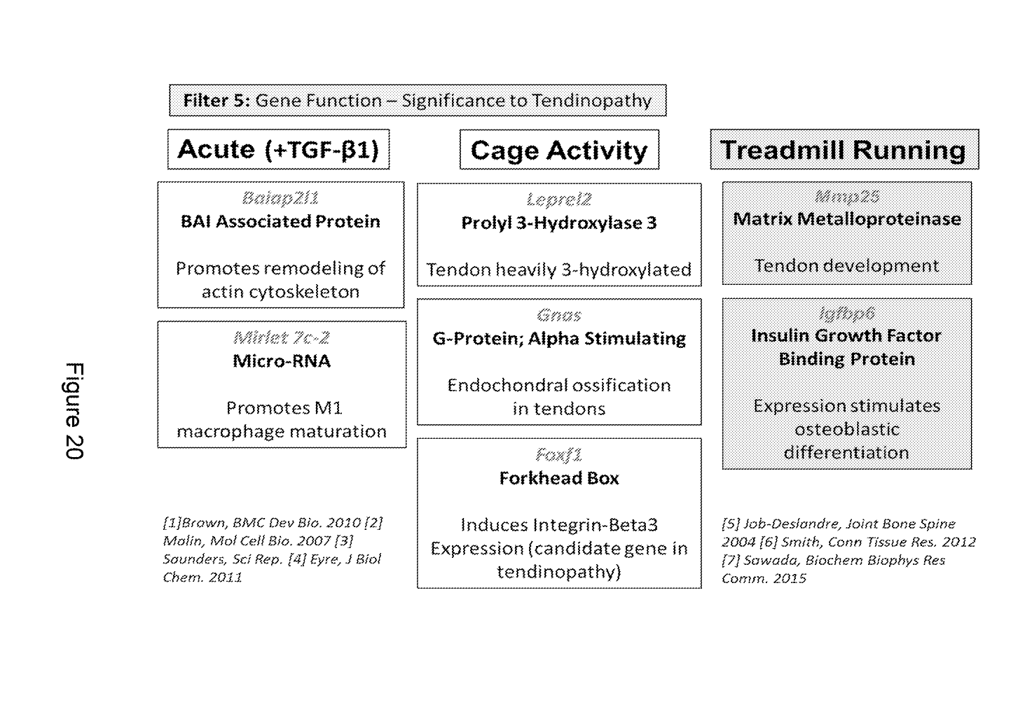

Finally as a different way to sort the methylation genes we can filter for genes that exhibit a direct connection to tendon biology and tendinopathy from our list of 22 genes which exhibit significantly differentially methylated sites. See FIG. 20. In direct response to TGF beta 1 at 3 days Baiap2l1 a brain angiogenesis inhibitor associated protein which promotes remodeling of the actin cytoskeleton and Mirelet7c-2 which is a micro-RNA that promotes macrophage maturation were identified. These are both indicative of an acute response to tendon injury. Looking at the cage active groups we've identified Foxf1, Leprel2, and Gnas. Foxf1 has been shown to induce Integrin beta 3 expression and interestingly this is 1 of 4 candidate genes in tendinopathy. Leprel2 is a proyl hydroxylase and rat tail tendon was found to be heavily 3-hydroxylated suggesting enhanced cross-linking and organization. Finally, Gnas gene has been linked to endochondral ossification in tendon. With treadmill running Mmp25 and Igfbp6 were identified. Increased levels of Mmp25 were found in the developing tendon of the mouse FDL and Igfbp6 has been shown to promote human peridontal ligament progenitor cells to an osteoblastic lineage.

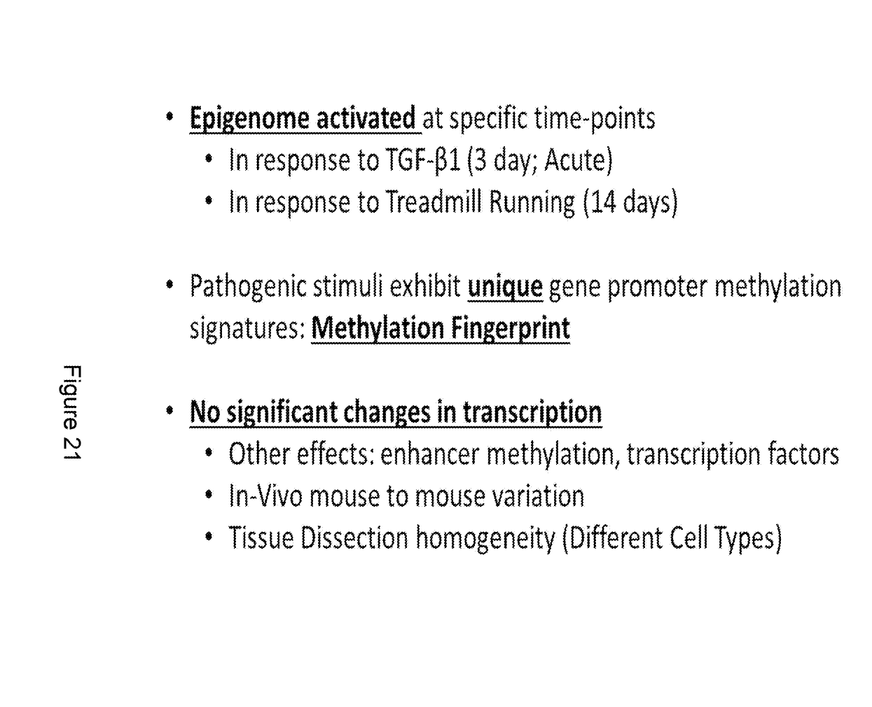

The epigenome is activated at specific time-points through expression of epigenetic enzymes (FIG. 21). Expression was highest in direct response to TGF beta 1 at the 3 day time-point or in response to treadmill running at 14 day. These pathogenic stimuli (TGF beta and treadmill running) exhibited unique gene promoter methylation signatures which could serve as a methylation fingerprint for the chronicity of tendinopathy.

Another aspect of the present invention provides methods for treating tendinopathy in a human or veterinary patient including administrating a therapy depending on a methylation change in a DNA methylome of the patient. For example, the change is a change in a promoter of at least one, or two, or three, or four, or five, or six, or seven of the genes listed in FIG. 18 (Baiap2l1, Cend1, Foxr1, Grm4, Gm19557, Mirlet7c-2, Rbmxl2, Forf1, Gnas, Hoxc4, Leprel2, Zrsr1, Igfbp6, Lfng, Mmp25, Sfi1, Bckdk, Cnot7, Meg3, Mir1199, Peg12, Rbmxl2, Shisa7 and Usp9x), or FIG. 19 (Baiap2l1, Cedn1, Rbmxl2, Lfng, Mm25, Sfi1 and Peg12) or FIG. 20 (Baiap2l1, Mirlet7c-2, Leprel2, Gnas, Foxf1, Mmp25, Igfbp6). In certain embodiments, the therapy is surgical reconstruction, physical therapy, viscosupplementation HA therapy, diet recommendations or life-style change recommendations. In other embodiments, the treatment includes administration of anti-inflammatory creams, gels or sprays, heat and freeze treatments, non-steroidal anti-inflammatory drugs (NSAIDs), acupuncture, complementary and alternative medicines, steroid injections or steroid tablets.

### Example 1—Murine Cartilage Injury Model

Hyaluronan Synthase 1 regulates macrophage activation during Joint Tissue Responses to Cartilage Injury. We investigate the role of macrophage activation after cartilage injury in the murine joint and evaluate potential cell sources within the joint of innate inflammatory responses. Activation of macrophages in an injured joint is regulated by hyaluronan synthase 1 (Has1). A proposed mechanism includes structural modification and turnover of HA-rich matrices and/or functional regulation of TLR and phagocytosis receptors. These studies underscore the importance for therapeutic manipulation of inflammatory responses of resident cell populations, especially in synovial tissue and subchondral bone, to aid the functional repair following traumatic joint injures.

Under an approved IACUC protocol, a non-bleeding cartilage injury was made in the right patellar groove of 10-12 week male C57Bl/6 wild type (WT) and Has1 knockout (Has1KO) mice [Chan, et al., Osteoarthritis Cartilage, 2015].

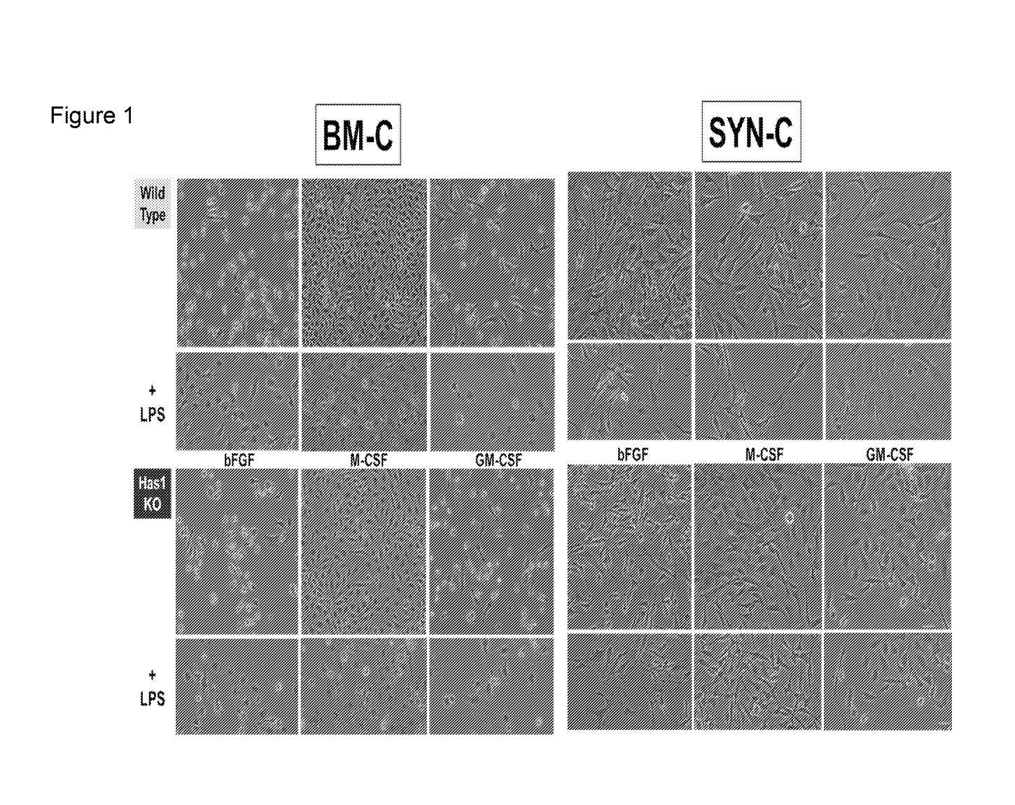

### Example 2—Bone Marrow and Synovium Derived Cell Cultures (FIG. 1)

Bone Marrow cell cultures (BM-C) were established [Yu, et al., J Exp Med, 2014. 211(5): p. 887-907] by plating 1×106 cells per well in 12 well plates in DMEM, 5 mM Glc/10% FCS supplemented with either 2 ng/mL rbFGF,10 ng/mL rM-CSF or 10 ng/mL rGMCSF. Non-adherent cells were removed after 24 h and cultured maintained for ˜6 days with 2 additional medium changes. For synovium derived cultures (SYN-C) perimeniscal synovium (combined from 10 mice) was digested for 1 h with collagenase (3 mg/mL) and isolated cells cultured as above for BM-C. Cultures were also treated at day 6 for 8 h with LPS (100 ng/mL in DMEM, 5 mM Glc/10% FCS).

### Example 3—Gene Expression

Taqman®-based qPCR was performed with inventoried probes (Applied Biosystems) for Gapdh, hyaluronan synthases 1 and 2 (Has1 & Has 2) and macrophage markers Arg1, Nos2, Emr1, and Itgam [Weisser, et al., Methods Mol Biol, 2013., Hamilton, et al., Front Immunol, 2014. 5: p. 554, He, et al., J Biol Chem, 2013.]. mRNA abundance relative to Gapdh was calculated as 1000×2^{-ΔCt}. For in vivo injured joints, two-way analysis of variance was performed with a significance level of a=0.05 and post hoc pairwise comparisons. Student's t test was used for in vitro experiments.

### Example 3—In Vivo Activation of Macrophage-Characteristic Genes Following Cartilage Injury

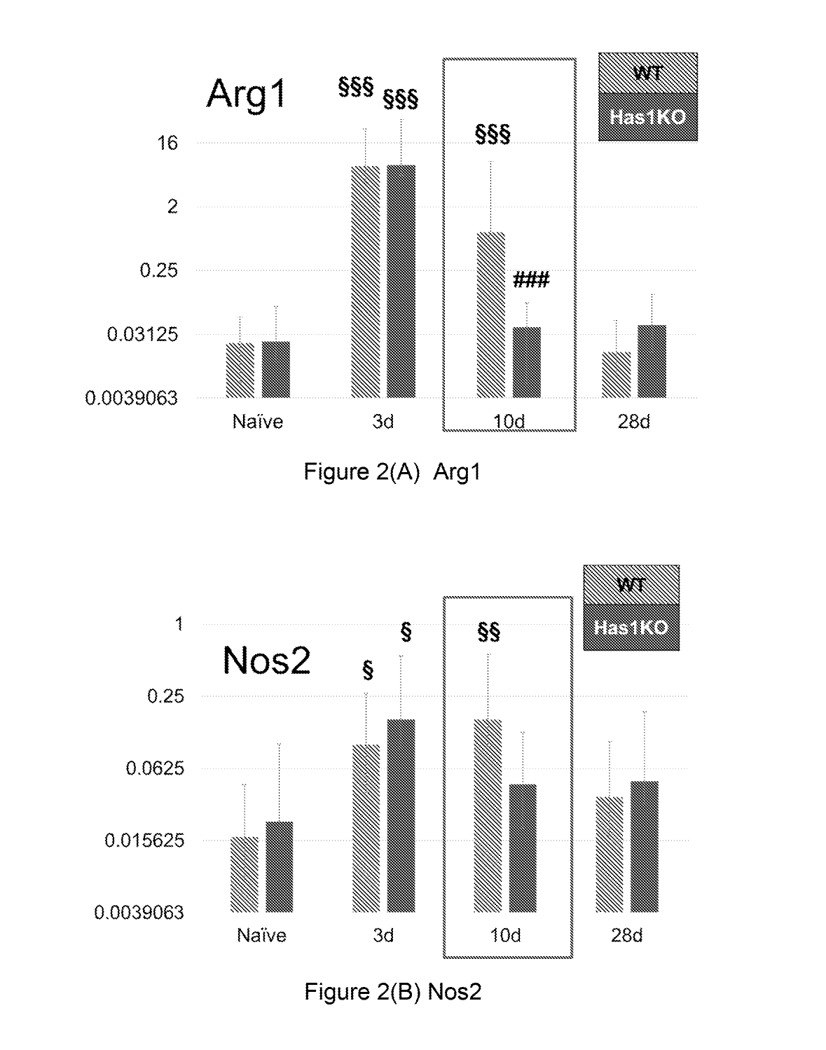

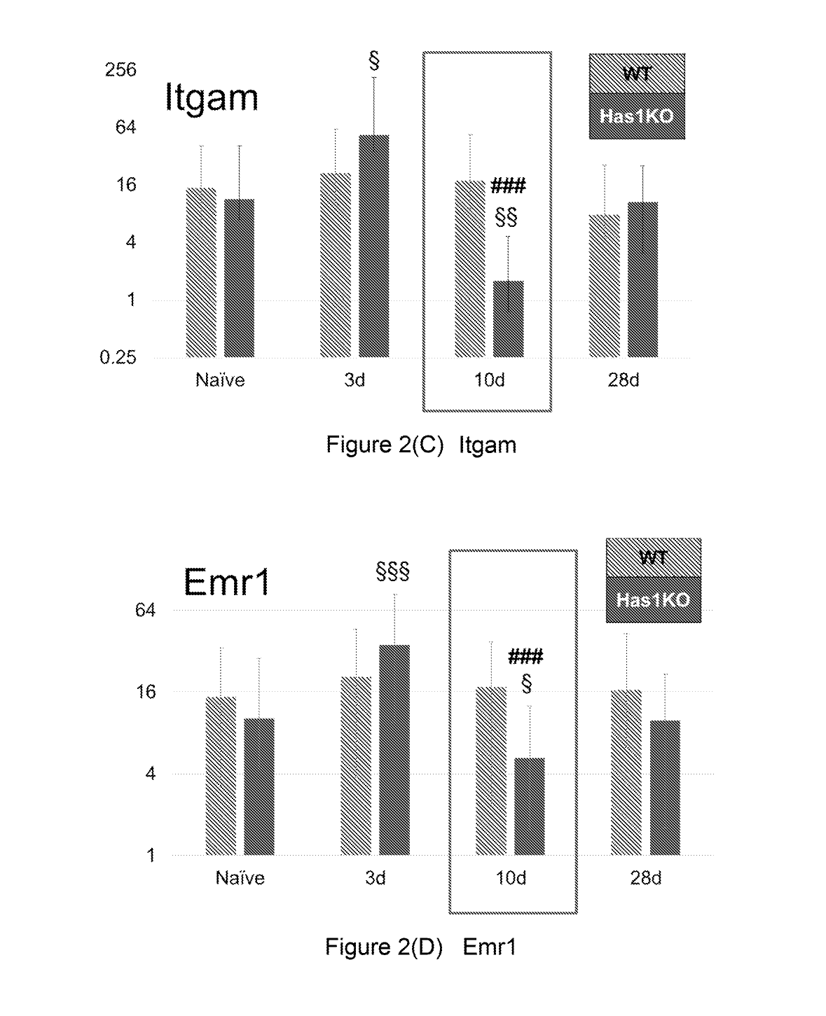

Expression of macrophage markers arginase 1 (Arg1) (FIG. 2(A)), inducible nitric oxide synthase 2 (Nos2) (FIG. 2(B)), Mac-1 (Itgam) (FIG. 2(C)), and F4/80 (Emr1) (FIG. 2(D)) was measured in naïve and injured WT and Has1KO mice and is shown as mean (±95% CI) mRNA abundance vs. Gapdh. P<0.05/0.01/0.001 for genotype time point vs. naïve (§/§§/§§§) and WT vs. Has1KO (#/##/###).

In WT joints, Arg1 and Nos2 expression was markedly increased at 3 days and remained elevated at 10 days. In Has1KO joints, Arg1 and Nos2 were also markedly increased at 3 days, however, in contrast to WT, their expression in Has1KO joints normalized by 10 days (see boxes). Expression of both enzymes was essentially normal by 28 days in both genotypes.

Most significantly, at 10 days the marked reduction of Nos2 and Arg1 in Has1KO joints was accompanied by a reduced expression (see red boxes) of both Itgam and Emr1.

### Example 4—Localization of Arginase 1 and NO Synthase 2 in Naïve and Injured Joints

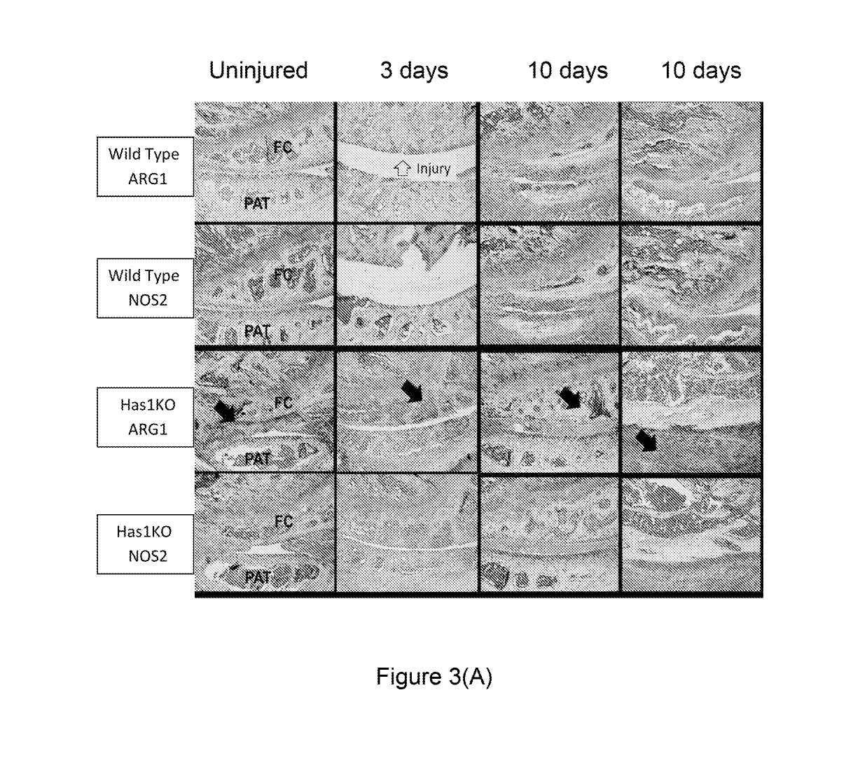

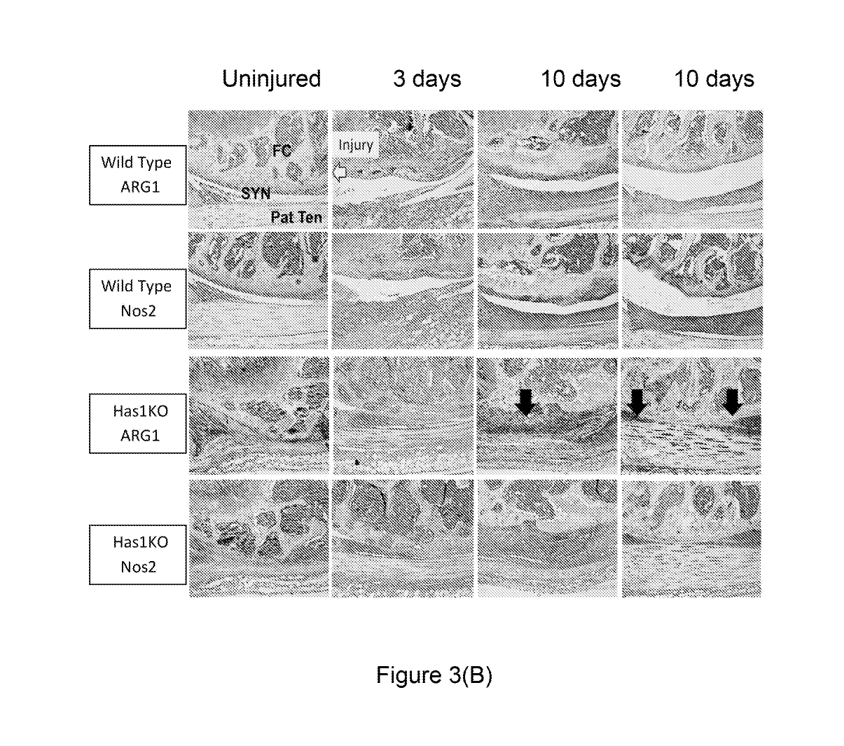

IHC Images of Patellofemoral (FIG. 3(A) and Peripatellar (FIG. 3B) Synovium/Subchondral Bone were obtained. The location of cells expressing NOS2 and ARG1 proteins was examined by IHC of thin sections of decalcified, paraffin-embedded whole joint sections. In uninjured WT and Has1KO joints, both ARG1 and NOS2 were detected in chondrocytes and synovial cells with ARG1 more abundant in Has1KO joints (FIG. 3(A)-3(B)—solid black arrows). There were no major changes in staining at 3 days post injury.

Most notably, at 10 days post injury very strong staining for ARG1 in bone marrow, synovium and patellar tendon of Has1KO joints only (solid black arrows) NOS2 staining was also still evident in those tissues with staining intensity similar in both genotypes. Both gene expression and IHC show a dysregulation of ARG1 (anti-inflammatory macrophage) in Has1KO joints relative to WT at 10 days. This suggests a mechanistic requirement of HAS1 protein in intrinsic repair processes after injury.

### Example 5—Effect of Macrophage Polarization by MCSF and GMCSF on Gene Expression in BM-C and SYN-C Cultures

Arg1 and Nos2 transcripts are low or below detection in unstimulated cells. Arg1, but not, Nos2 is stimulated by both MCSF (FIG. 4(B)) and GM-CSF (FIG. 4(C)) priming but is more pronounced in SYN-C compared to BMS-C. This stimulatory effect is less pronounced in Has1KO compared to WT cells

### Example 6—Effect of Macrophage Stimulation by LPS on Arg1 and Nos2 Gene Expression in BM-C and SYN-C Culture

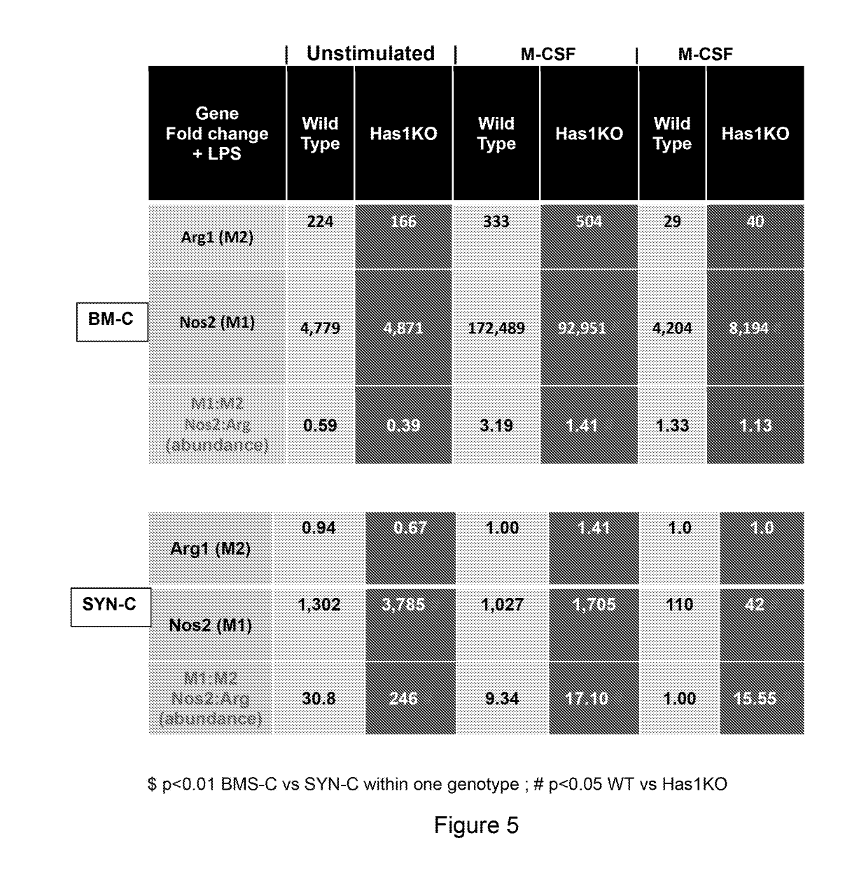

LPS addition resulted in a robust stimulation of Nos2 expression in both, BMS-C and SYN-C cultures (FIG. 5). However, for Has1KO BM-C cultures, that had pretreated with MCSF or GMCSF, the magnitude of induction was significantly lower than in WT cells. LPS also increased Arg1 transcripts, but only in BM-C cultures. The ratio of transcripts for Nos2:Arg1 as an index of M1 (inflammatory) vs M2 (anti-inflammatory) polarization suggest that in SYN-C (but not BM-C), LPS stimulation results in the predominant activation of the inflammatory macrophage phenotype. Interestingly, this enhancement of the pro-inflammatory type was more pronounced in unstimulated and MCSF or GMCSF-primed Has1KO cultures

### Example 7—Tendon Explant Culture Methodologies

Tendon explant culture methodologies allow for mechanistic studies of tendon cell metabolism independent of contributions from surrounding tissues, while maintaining cells in their native extracellular matrix (ECM). Explant culture conditions (e.g. media composition, time) for murine Achilles tendons can result in preservation of the structural and biomechanical properties. (Trella+. Trans Orthop Res Society. 2013.) We examined the effects of altered oxygen (O₂) concentration and TGFb1 on the cellular responses of murine Achilles tendons in explant culture.

All experiments were performed under approved IACUC protocols using 12-wk C57BL/6 male mice. Naïve (uninjured/uncultured) mice are compared with in-vivo injured mice [Bell+J Biomech 2013]. These mice received two injections (2 days apart) of 6 pL of 100 ng rhTGFb1 into the Achilles tendon body. The mice sacrificed and tendons dissected from all surrounding tissue after 3 days.

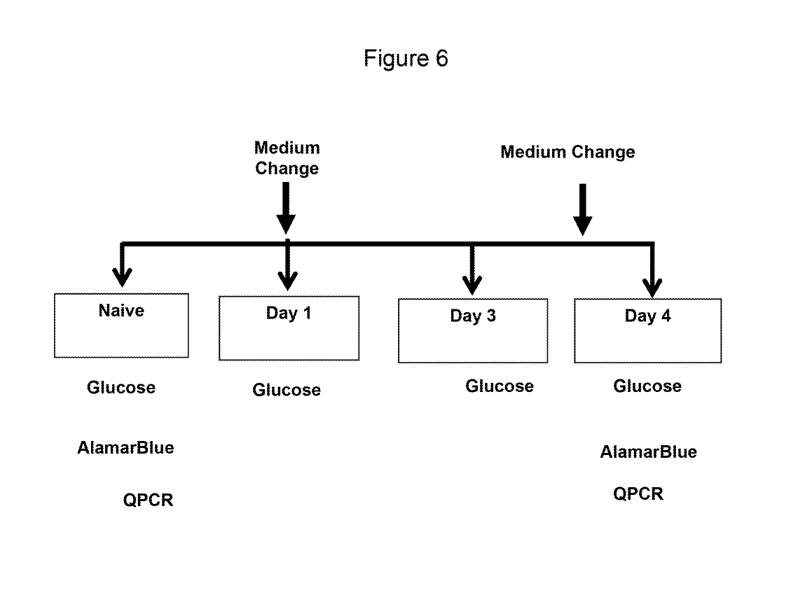

Tendons were dissected into CO₂ Independent MEM (on ice). Explants were cultured under free-floating conditions in AMEM (1% FBS), 5 mM glucose, 5 mM glucosamine, and 1% glutamine (1 mL media/tendon) using the protocol illustrated in FIG. 6. See also Trella+. Trans. Orthop. Res. Society. 2013. The following conditions were included:
High Oxygen (20% O₂)
Low Oxygen (2.5% O₂)
High Oxygen (20% O₂)+10 ng/mL TGFb1
Low Oxygen (2.5% O₂)+10 ng/mL TGFb1

### Example 8—Expression of ECM and Hypoxia Signaling Genes

RNA was purified from 20 pooled tendons per group using the method of Bell+ J Biomech 2013. Taqman QPCR assays were performed for Col1a1, Col2a1, Col3a1, Acan, and Mmp3 using SYBR Hypoxia signaling arrays (PAMM-032ZA, Qiagen). The array contains 81 genes from 11 functional groups: Hif1a and co-transcription factors, other Hif1a interactors, angiogenesis, coagulation, DNA damage signaling and repair, metabolism, apoptosis, cell proliferation, transcription factors, transporters/receptors, other responsive genes. Data is calculated as fold change relative to naïve (2̂−ΔΔCt). The Housekeeping Gene is B2m and >2-fold considered biologically significant.

FIG. 7 shows the fold change in expression of ECM genes. FIG. 8 shows the percentage of hypoxia signaling genes up or down-regulated. Explant cultures in Low O₂ and/or with TGFb1 resulted in up-regulated expression of matrix proteins (FIG. 7) and hypoxia signaling genes (FIG. 8) as compared to culture. Low O₂ and/or with TGFb1 resulted in expression of genes involved in metabolism as seen after in-vivo injury of the tendon (FIG. 9).

### Example 9—AlamarBlue Assay of Intracellular Reducing Equivalents (NADH/NADPH) Generated by Glycolysis and TCA Cycle Activity

4-6 tendons per group are placed individually into 1 mL fresh culture medium and 10% (v/v) alamarBlue (Invitrogen) and incubated at 37° C. for 24 hours in 20% O₂. The media is removed and fluorescence measured (ex:530 nm, em:590 nm) against a ‘medium blank’ (no tissue). Statistically significant changes relative to uncultured naïve tissue are determined by 1-way ANOVA followed by Tukey's post-hoc tests (*p<0.05) using GraphPad Prism 5 (La Jolla, Calif.). Explant culture in High O₂ resulted in significantly elevated levels of NADH/NADPH reducing activity as seen after in-vivo injury of the tendon. (FIG. 10).

### Example 10—Glucose Uptake and Gluconeogenesis in Explant Cultures

The medium is removed at day 1, 3, and 4 and assayed for glucose content using the Amplex™ kit (LifeTech Inc.) Concentration is calculated from blank controls (day 0), and expressed as μmol of glucose in medium (per tendon). Explant culture in High O₂ results in reduction of medium glucose at 1 and 3 days, whereas all other conditions results in an increase, consistent with induction of gluconeogenesis (from glycogen and/or glucogenic amino acids). See FIG. 11.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A method for classifying the quality of a repair response after injury to a joint of a human or veterinary patient, comprising:
determining mRNA expression levels (transcriptosome) of at least two of a plurality of genes listed in FIG. 4(A) expressed in a sample from the patient; and
calculating a reparative index score based on the mRNA expression levels of the at least two of the plurality of genes,
wherein the reparative index score is indicative of the quality of the repair process.

2. The method of claim 1, wherein the sample is a tissue sample taken from an intra- or peri-articular region of the joint.

3. The method of claim 1, wherein the sample is a blood sample or synovial fluid aspirate, containing cells.

4. The method of claim 1, comprising determining mRNA expression levels of at least three of a plurality of genes listed in FIG. 4(A) expressed in a sample from the patient; and calculating a reparative index score based on the mRNA expression levels of the at least three of the plurality of genes.

5. The method of claim 4, comprising determining mRNA expression levels of at least four of a plurality of genes listed in FIG. 4(A) expressed in a sample from the patient; and calculating a reparative index score based on the mRNA expression levels of the at least four of the plurality of genes.

6. A method for characterizing tendinopathy in a patient, comprising detecting a methylation change in a DNA methylome of the patient, wherein the change is indicative of the tendinopathy.

7. The method of claim 6, wherein the change is a change in a promoter of at least one of the genes listed in FIG. 18.

8. The method of claim 7, wherein the change is a change in a promoter of at least three of the genes listed in FIG. 18, wherein the method further comprises calculating an index score based on the change in the promoter of the at least three of the genes listed in FIG. 18, and wherein the index score is indicative of the tendinopathy.

9. The method of claim 6, wherein the change is a change in a promoter of at least one of the genes listed in FIG. 19.

10. The method of claim 9, wherein the change is a change in a promoter of at least three of the genes listed in FIG. 19, wherein the method further comprises calculating an index score based on the change in the promoter of the at least three of the genes listed in FIG. 19, and wherein the index score is indicative of the tendinopathy.

11. The method of claim 6, wherein the change is a change in a promoter of at least one of the genes listed in FIG. 20.

12. The method of claim 11, wherein the change is a change in a promoter of at least three of the genes listed in FIG. 20, wherein the method further comprises calculating an index score based on the change in the promoter of the at least three of the genes listed in FIG. 20, and wherein the index score is indicative of the tendinopathy.

13. A method of tendon explant culture methodology allowing for mechanistic studies of tendon cell metabolism independent of contributions from surrounding tissues, while maintaining cells in their native extracellular matrix.

14. The method of claim 13, comprising measuring expression of ECM and hypoxia signaling genes during explant culture in High O₂.

15. The method of claim 13 comprising measuring expression levels of at least two of the genes listed in FIG. 7.
